(19) United States
(12) Patent Application Publication
(10) Pub. No.: US 2024/0079103 A1
(43) Pub. Date: Mar. 7, 2024

(54) INFORMATION PROCESSING DEVICE, INFORMATION PROCESSING METHOD, AND INFORMATION PROCESSING SYSTEM

(71) Applicant: SONY GROUP CORPORATION, TOKYO (JP)

(72) Inventors: RITSUKO KANO, TOKYO (JP); EIJIRO MORI, TOKYO (JP); SHINSUKE NOGUCHI, TOKYO (JP); KAZUMI HIRANO, TOKYO (JP); TAKAFUMI YANAGIMOTO, TOKYO (JP); KOJI SATO, TOKYO (JP); HIROSHI HARA, TOKYO (JP)

(21) Appl. No.: 18/262,052

(22) PCT Filed: Dec. 23, 2021
(86) PCT No.: PCT/JP2021/047765

§ 371 Date: Jul. 19, 2023

(30) Foreign Application Priority Data
Jan. 27, 2021 (JP) 2021-011218

(51) Int. Cl. G16H 10/60; G16H 20/10

(52) U.S. Cl. CPC G16H 10/60; G16H 20/10

(57) ABSTRACT

An information processing device (40) according to one embodiment of the present disclosure includes: an analysis unit (41) being a first data generation unit that generates objective score data, which indicates objective scores in time series, based on a plurality of pieces of objective data regarding a patient; a processing unit (42) being a second data generation unit that generates subjective score data, which indicates subjective scores in time series, based on a plurality of pieces of subjective data obtained from the patient; and a generation unit (44) being an image generation unit that generates a score image indicating the objective score data and the subjective score data.

### FIELD

The present disclosure relates to an information processing device, an information processing method, and an information processing system.

### BACKGROUND

Currently, regarding chronic diseases such as asthma, allergic diseases, and diabetes, recording of patient state sensing information (such as blood pressure, blood glucose level, behavior, for example) is performed, as well as a medical inquiry with several questions. This has led to development of techniques related to means of obtaining patient sensing information and means of conducting the medical inquiry. Typically, the clinician decides a treatment policy based on the sensing information and a result of the medical inquiry described above.

### CITATION LIST

### Patent Literature

Patent Literature 1: JP 2020-13245 A

### SUMMARY

### Technical Problem

However, the above-described technology focuses on a way to easily obtain the sensing information and the result of medical inquiry. Therefore, the recording of the sensing information and the medical inquiry are independent from each other, making it difficult to use these pieces of information comprehensively for communication between the clinician and the patient, and difficult to be linked to the treatment incorporating patient's subjective feeling. In addition, the verbal discrepancy between the clinician and the patient and a shortage of medical examination time with the clinician make it difficult for the patient to accurately express their symptoms and to accurately understand the words of the clinician, leading to an occurrence of a communication gap between the clinician and the patient.

This can lead to inappropriateness of the clinician's treatment policy, and inappropriate treatment of the patient, resulting in no improvement of the symptom in some cases. Although a chronic disease needs continuous treatment, there may be a case where the treatment cannot be continued due to the reason such as interruption of medication on patient's self-judgment. This leads to a demand for a technique for supporting continuation of treatment.

In view of this, the present disclosure proposes an information processing device, an information processing method, and an information processing system capable of supporting continuation of treatment.

### Solution to Problem

An information processing device according to the embodiment of the present disclosure includes: a first data generation unit that generates objective score data, which indicates objective scores in time series, based on a plurality of pieces of objective data regarding a patient; a second data generation unit that generates subjective score data, which indicates subjective scores in time series, based on a plurality of pieces of subjective data obtained from the patient; and an image generation unit that generates a score image indicating the objective score data and the subjective score data.

An information processing method according to the embodiment of the present disclosure performed by a computer, the method includes: generating objective score data, which indicates objective scores in time series, based on a plurality of pieces of objective data regarding a patient; generating subjective score data, which indicates subjective scores in time series, based on a plurality of pieces of subjective data obtained from the patient; and generating a score image indicating the objective score data and the subjective score data.

An information processing system according to the embodiment of the present disclosure includes: a patient terminal device that transmits a plurality of pieces of objective data regarding a patient and a plurality of pieces of subjective data obtained from the patient; a first data generation unit that generates objective score data, which indicates objective scores in time series, based on the plurality of pieces of objective data transmitted by the patient terminal device; a second data generation unit that generates subjective score data, which indicates subjective scores in time series, based on the plurality of pieces of subjective data transmitted by the patient terminal device; an image generation unit that generates a score image indicating the objective score data and the subjective score data; and a display unit that displays the score image generated by the image generation unit.

### BRIEF DESCRIPTION OF DRAWINGS

FIG. 1 is a first diagram illustrating an example of a schematic configuration of an information processing system according to an embodiment of the present disclosure.

FIG. 2 is a second diagram illustrating an example of a schematic configuration of the information processing system according to the embodiment of the present disclosure.

FIG. 3 is a flowchart illustrating an example of a flow of data processing according to the embodiment of the present disclosure.

FIG. 4 is an explanatory diagram illustrating an example of objective data acquisition according to the embodiment of the present disclosure.

FIG. 5 is a first explanatory diagram illustrating an example of subjective data acquisition according to the embodiment of the present disclosure.

FIG. 6 is a second explanatory diagram illustrating an example of subjective data acquisition according to the embodiment of the present disclosure.

FIG. 7 is a third explanatory diagram illustrating an example of subjective data acquisition according to the embodiment of the present disclosure.

FIG. 8 is a fourth explanatory diagram illustrating an example of subjective data acquisition according to the embodiment of the present disclosure.

FIG. 9 is a first explanatory diagram illustrating an example of visualization of objective data and subjective data according to the embodiment of the present disclosure.

FIG. 10 is a second explanatory diagram illustrating an example of visualization of objective data and subjective data according to the embodiment of the present disclosure.

FIG. 11 is a third explanatory diagram illustrating an example of visualization of objective data and subjective data according to the embodiment of the present disclosure.

FIG. 12 is a fourth explanatory diagram illustrating an example of visualization of objective data and subjective data according to the embodiment of the present disclosure.

FIG. 13 is a first explanatory diagram illustrating an example of support of continuous treatment according to the embodiment of the present disclosure.

FIG. 14 is a second explanatory diagram illustrating an example of support of continuous treatment according to the embodiment of the present disclosure.

FIG. 15 is a third explanatory diagram illustrating an example of support of continuous treatment according to the embodiment of the present disclosure.

FIG. 16 is a first explanatory diagram illustrating an example of a screen according to the embodiment of the present disclosure.

FIG. 17 is a second explanatory diagram illustrating an example of a screen according to the embodiment of the present disclosure.

FIG. 18 is a third explanatory diagram illustrating an example of a screen according to the embodiment of the present disclosure.

FIG. 19 is a fourth explanatory diagram illustrating an example of a screen according to the embodiment of the present disclosure.

FIG. 20 is a fifth explanatory diagram illustrating an example of a screen according to the embodiment of the present disclosure.

FIG. 21 is a first explanatory diagram illustrating an example of medication management according to the embodiment of the present disclosure.

FIG. 22 is a second explanatory diagram illustrating an example of medication management according to the embodiment of the present disclosure.

FIG. 23 is a diagram illustrating an example of a hardware schematic configuration according to the embodiment of the present disclosure.

### DESCRIPTION OF EMBODIMENTS

Embodiments of the present disclosure will be described below in detail with reference to the drawings. Note that the information processing device, the information processing method, and the information processing system according to the present disclosure are not limited by the embodiments. Moreover, basically in each of the following embodiments, the same parts are denoted by the same reference symbols, and a repetitive description thereof will be omitted.

One or more embodiments (examples and modifications) described below can each be implemented independently. On the other hand, at least some of the plurality of embodiments described below may be appropriately combined with at least some of other embodiments. The plurality of embodiments may include novel features different from each other. Accordingly, the plurality of embodiments can contribute to achieving or solving different objects or problems, and can exhibit different effects.

The present disclosure will be described in the following order.
1. Introduction
2. Embodiments
2-1. Schematic configuration of information processing system
2-2. Data processing flow
2-3. Objective data related to objective evaluation values
2-4. Subjective data related to subjective evaluation values
2-5. Visualization of objective data and subjective data
2-6. Supporting continuous treatment
2-7. Screen example
2-8. Medication management
2-9. Effects
3. Other embodiments
4. Hardware configuration example
5. Supplementary notes

### 1. Introduction

Currently, there have been developed a technique including recording of patient state sensing information (such as blood pressure, blood glucose level, behavior, for example), medical inquiry with several questions, and indicating the relevance between environmental information and symptoms. However, even with such data, it is difficult in some cases to fill the communication gap between the clinician and the patient, and a lack of appropriate treatment might result in no improvement of the symptoms. For example, the verbal discrepancy between the clinician and the patient and the shortage of examination time that can be taken by the clinician make it difficult for the patient to accurately express the symptoms and to accurately understand the words of the clinician, leading to miscommunication between the clinician and the patient. In the medical inquiry, the patient relies on their past memories and tends to answer like “I get the feeling that it happened”. This similarly applies to the occurrence that happened on the same day of examination. In recent years, treatment for the purpose of improving quality of life (QoL) focusing on the patients has also been promoted. However, the medical inquiry just for acquiring QoL is far from accurately understanding the patient's feeling.

In addition, while there is a need to continuously perform the medical inquiry with the patient, it is difficult to continuously perform medical inquiry. One of the factors of this problem is a high input load on the user being a patient. In addition, the vagueness of the benefit of inputting an answer to the medical inquiry is also one of the factors to lower the motivation. In a chronic disease in particular, the patient often needs to take a medication continuously even with no symptoms. However, due to the situation in which the patient misses a dose or stops taking the medication by own judgment, the symptoms do not improve in some cases. Therefore, there is another problem of difficulty in supporting the continuation of medication.

In view of these, in order to fill a communication gap during treatment between a clinician and a patient, an embodiment of the present disclosure provides a system that supports continuation of treatment by implementing techniques such as visualization of symptoms of the patient. This system combines a plurality of pieces of objective data obtained from sensing information such as biometric information with a plurality of pieces of subjective data such as a medical inquiry result, for example. That is, the system to be provided is a system that appropriately visualizes data acquired as a communication language between a clinician and a patient so as to enable the patient of a chronic disease to maintain treatment continuation motivation and to be useful for symptom control.

For example, the system acquires data related to a symptom of a patient with a chronic disease, specifically, each piece of objective data regarding a patient and each piece of subjective data of the patient, displays these pieces of data in time series in the form of an objective score and a subjective score, and appropriately transmits information useful for symptom control to the patient, a clinician, a medical worker, and the like, thereby enabling support of continuation of treatment. In addition, continuation of treatment can be supported by simultaneously performing medication management and the like. That is, using information based on each piece of objective data regarding the patient and each piece of subjective data obtained from the patient, a common language between the clinician and the patient is provided. The treatment smoothly proceeds using the common language, making it possible to achieve improvement of the symptom. In addition, by visualizing the treatment status and the symptom status while performing medication management, improvement or aggravation in the state of the symptom can be revealed, making it possible to achieve appropriate therapeutic intervention by a clinician.

Note that data processing includes usage of a means of reducing the number of dimensions of the plurality of pieces of acquired objective data and the plurality of pieces of acquired subjective data. For example, each objective data, which is vital signs data obtained by sensing, is integrated as an objective score, while each subjective data obtained by patient input is integrated as a subjective score. In addition, both the objective score data and the subjective score data are visualized and used as a communication language (tool) between the clinician and the patient. For example, the objective score obtained from each objective data and the subjective score obtained from each subjective data are visualized in a map (in the form of a graph or the like) in time series so as to visualize the fluctuations of the scores. In addition, the point with a large change or the total score up to a certain time point is presented and appropriately verbalized. Details will be described in each embodiment.

### 2. Embodiments

### 2-1. Schematic Configuration of Information Processing System

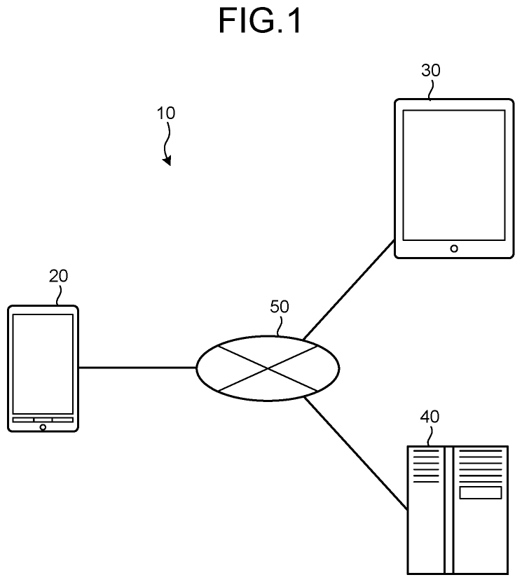

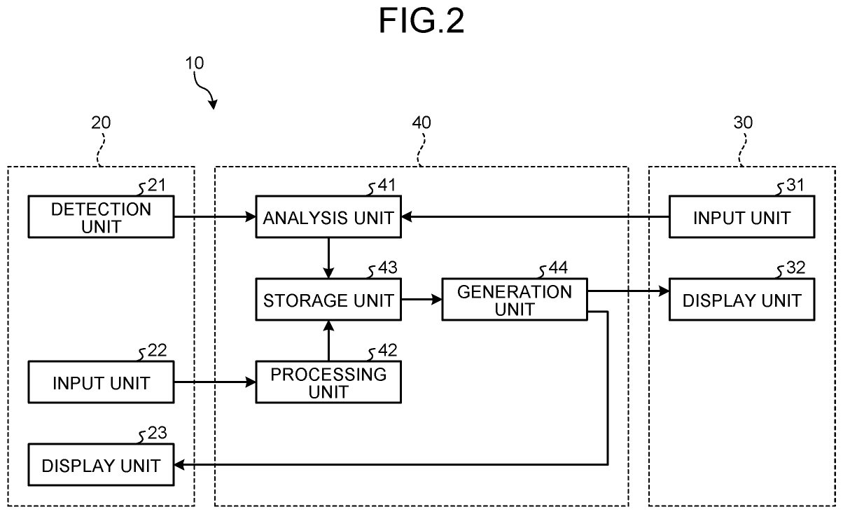

An example of a schematic configuration of an information processing system 10 according to the present embodiment will be described with reference to FIGS. 1 and 2. FIGS. 1 and 2 are diagrams illustrating an example of a schematic configuration the information processing system 10 according to the present embodiment. In the examples of FIGS. 1 and 2, the information processing system 10 functions as a treatment continuation support system that supports continuation of treatment.

As illustrated in FIG. 1, the information processing system 10 includes a patient terminal device 20, a clinician terminal device 30, and an information processing device 40. Various types of information are transmitted and received among the patient terminal device 20, the clinician terminal device 30, and the information processing device 40. This transmission and reception are executed via a communication network 50 using both or one of wireless and wired communications.

As illustrated in FIG. 2, the patient terminal device 20 includes a detection unit 21, an input unit 22, and a display unit 23. The patient terminal device 20 is used by a patient. The detection unit 21 detects various types of information such as biometric information and environmental information. The detection unit 21 is implemented by, for example, a sensor such as a vital signs sensor, a camera, a microphone, or an acceleration sensor. The input unit 22 receives an input operation from an operator such as a patient. The input unit 22 is implemented by, an input device such as a touch panel and a button, for example. The display unit 23 displays various types of information. The display unit 23 is implemented by a display device such as a liquid crystal display and an organic electro-luminescence (EL) display, for example. The input unit 22 may be implemented by a voice input device (for example, a microphone) that receives an operator's input operation using voice.

The patient terminal device 20 transmits a plurality of pieces of detection information (such as biometric information and environmental information, for example) detected by the detection unit 21 and a plurality of pieces of input information (such as information concerning answer to the medical inquiry, for example) input by the patient to the information processing device 40. The detection information detected by the detection unit 21 corresponds to objective data regarding the patient. Furthermore, the input information input by the patient corresponds to subjective data of the patient. Note that the detection unit 21 includes various sensors, and can obtain various types of detection information (sensing data).

Applicable examples of such a patient terminal device 20 include a wearable device of a wristband type, a neckband type, or an earphone type, or a device such as a smartphone. The wearable device and the smartphone include an acceleration sensor, a microphone, and a sensor capable of acquiring various types of vital signs data such as a pulse wave and skin perspiration.

As illustrated in FIG. 2, the clinician terminal device 30 includes an input unit 31 and a display unit 32. The clinician terminal device 30 is used by a clinician (or a medical worker). The input unit 31 receives an input operation from an operator such as a clinician. The input unit 31 is implemented by, for example, an input device such as a touch panel or a button. The display unit 32 displays various types of information. The display unit 32 is implemented by a display device such as a liquid crystal display and an organic electro-luminescence (EL) display, for example. The input unit 31 may be implemented by a voice input device (for example, a microphone) that receives an operator's input operation using voice.

As illustrated in FIG. 2, the information processing device 40 includes an analysis unit 41, a processing unit 42, a storage unit 43, and a generation unit 44. The information processing device 40 functions as a centralized signal processing device (an example of a server). The analysis unit 41 corresponds to a first data generation unit, the processing unit 42 corresponds to a second data generation unit, and the generation unit 44 corresponds to an image generation unit.

The analysis unit 41 generates objective score data indicating objective scores in time series based on a plurality of pieces of objective data (such as biometric information, and environmental information, for example) transmitted by the patient terminal device 20. For example, the analysis unit 41 integrates each objective data into objective scores to be arranged in time series, thereby generating objective score data (details will be described below).

The processing unit 42 generates subjective score data indicating the subjective scores in time series based on a plurality of pieces of subjective data (such as information concerning answer to the medical inquiry, for example) transmitted by the patient terminal device 20. For example, the processing unit 42 integrates each subjective data into subjective scores to be arranged in time series, thereby generating subjective score data (details will be described below).

The storage unit 43 stores various data such as objective score data and subjective score data. The storage unit 43 is implemented by semiconductor memory elements such as flash memory, or storage devices such as a hard disk or an optical disk. Note that various data generated by the analysis unit 41, the processing unit 42, the generation unit 44, and the like are stored in the storage unit 43 as necessary.

Based on the data such as the objective score data and the subjective score data stored by the storage unit 43, the generation unit 44 generates a score image indicating the objective score data and the subjective score data (details will be described below). An applicable example of the score image is a graph (map) indicating each data in time series.

Here, each functional unit such as the analysis unit 41, the processing unit 42, the storage unit 43, and the generation unit 44 described above may be implemented by both or any one of hardware and software configurations. These configurations are not particularly limited. Furthermore, the analysis unit 41 and the processing unit 42 may be integrated as an analysis unit or a processing unit. For example, each of the above-described functional units may be implemented by execution of a program stored in advance in the ROM by a computer such as a central processing unit (CPU) or a micro control unit (MPU) using RAM or the like as a work area. In addition, each of the functional units may be implemented by an integrated circuit such as an application specific integrated circuit (ASIC) or a field-programmable gate array (FPGA).

According to such an information processing system 10, a change in daily symptoms of a patient is acquired by automatic sensing and subjective symptom evaluation (for example, medical inquiry) from the patient, and the acquired data (objective data regarding the patient and subjective data obtained from the patient) is visualized as a score, making it possible to eliminate the communication gap by using the acquired data as a communication language (or tool) between the clinician and the patient.

For example, based on data acquired from the patient terminal device 20, the information processing device 40 automatically generates a score image functioning as a symptom visualization map as a communication language between a clinician and a patient. Source data of the score image is to be objective data related to an objective evaluation value by sensing or environmental information as well as subjective data related to a patient's subjective evaluation value. For example, this application is targeted to a disease in which subjective evaluation such as QoL of a patient contributes to understanding of symptoms at a high rate and improvement of symptoms is not to be obtained only by an objective evaluation value.

Furthermore, by displaying a prospect of future symptom improvement on the symptom visualization map, for example, the information processing device 40 can motivate the patient to continue the treatment and achieve successful continuation of the treatment. Furthermore, by automatically acquiring the medication status of the patient under treatment, it is also possible to prevent a missed dose and assist continuous treatment.

Here, the information processing device 40 described above may be provided in a hospital or may be provided outside the hospital. The information processing device 40 functions as a server device, but may be implemented by cloud computing. Furthermore, the patient terminal device 20 and the clinician terminal device 30 may be implemented by a portable terminal such as a smartphone or a tablet, or may be implemented by a personal computer or the like. Furthermore, the patient terminal device 20 may have the detection unit 21 as a unit separate from the device.

### 2-2. Data Processing Flow

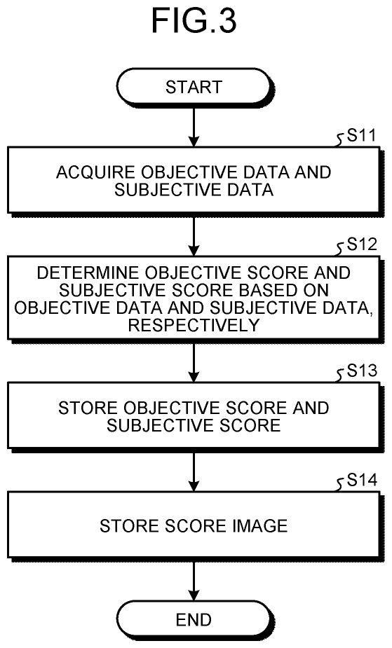

An example of a data processing flow according to the present embodiment will be described with reference to FIG. 3. FIG. 3 is a flowchart illustrating an example of a data processing flow that is, a score image generation processing flow according to the present embodiment.

As illustrated in FIG. 3, the information processing device 40 acquires a plurality of pieces of objective data and a plurality of pieces of subjective data from the patient terminal device 20 which is a user terminal (step S11). The patient terminal device 20 transmits each objective data (such as biometric information and environmental information, for example) and each subjective data (such as information concerning answer to the medical inquiry, for example) to the information processing device 40 via the communication network 50. The information processing device 40 receives and acquires each objective data and each subjective data transmitted from the patient terminal device 20.

The information processing device 40 uses the analysis unit 41 to determine an objective score from each objective data, and uses the processing unit 42 to determine a subjective score (step S12). For example, the analysis unit 41 performs dimensionality reduction on each objective data based on predetermined weighting and determines an objective score. Similarly, the processing unit 42 also performs dimensionality reduction on each subjective data based on predetermined weighting and determines a subjective score. Note that the dimensionality reduction processing by weighting will be described below in detail.

Next, the information processing device 40 stores the objective score and the subjective score by the storage unit 43 (step S13). For example, the information processing device 40 acquires an objective score and a subjective score each time each objective data and/or each subjective data is acquired from the patient terminal device 20, and stores the acquired objective score and subjective score in association with date and time. That is, the information processing device 40 chronologically acquires and stores the objective score and the subjective score. With this operation, the objective score data indicating the subjective score in time series and the subjective score data indicating the subjective score in time series are stored in the storage unit 43. These pieces of data are stored in association with a patient (represented by patient identification information such as a name and a patient number, for example) and managed for each patient. Note that other data regarding the patient is also stored in association with the patient and managed for each patient.

Next, based on the objective score data and the subjective score data stored by the storage unit 43, the information processing device 40 generates a score image such as a graph for treatment support (step S14). This score image is transmitted to the patient terminal device 20 or the clinician terminal device 30 in response to a request from the patient terminal device 20 or the clinician terminal device 30, for example. The patient terminal device 20 acquires the score image from the information processing device 40 and displays the score image on the display unit 23. In addition, the clinician terminal device 30 also acquires the score image from the information processing device 40 and displays the score image on the display unit 32. Note that the patient can display only their own score image on the display unit 23, and the clinician can select a score image to be viewed from among the score images for individual patients and display the selected score image on the display unit 32.

### 2-3. Objective Data Related to Objective Evaluation Values

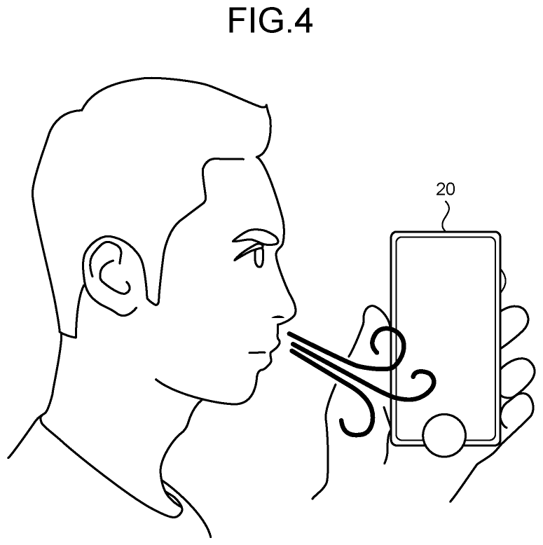

An example of objective data regarding an objective evaluation value according to the present embodiment will be described with reference to FIG. 4. FIG. 4 is an explanatory diagram illustrating an example of objective data acquisition according to the present embodiment.

### First Example of Objective Data

Using the detection unit 21 such as a vital signs sensor, the patient terminal device 20 detects and acquires data including a pulse wave, a heart rate, a body temperature, skin perspiration, and oxygen saturation (SpO2) as vital signs data which is an example of objective data. Non-invasive sensing is performed as much as possible in order to reduce the load on the patient. However, it is also possible to use data by invasive sensing depending on the disease. In the treatment of each disease, an accurate value can be acquired by using dedicated equipment. Alternatively, it is also allowable to acquire vital signs data unique to a disease and particularly noteworthy in a simplest form and grasp a change in the state of a patient by focusing on the change even with the value in the simple form.

For example, in the case of a disease of asthma, a dedicated device referred to as a peak flow meter that measures the expiratory volume per unit time is usually used. In the present embodiment, the patient terminal device 20 is used to monitor the change in the state of the patient. For example, as illustrated in FIG. 4, when a patient blows a breath to the patient terminal device 20 such as a smartphone, which is a familiar device having a microphone function, the patient terminal device 20 acquires a sound of the patient's blown breath by the detection unit 21 such as a microphone and transmits sound information to the information processing device 40. In response to this, based on the sound information acquired from the patient terminal device 20, the information processing device 40 uses the analysis unit 41 to determine a change in the symptom of asthma (an example of the symptom of the patient) based on the loudness of sound (the magnitude of sound), the amount of noise (for example, wheezing) included in the sound, the duration of the sound, and the like, and generates expiratory information regarding the expiratory volume (an example of the symptom information of the patient) to be recorded in the storage unit 43. This expiratory information is used as objective data.

In addition, it is possible to determine, from the difference in tone of voice in the patient's speech, emotional ups and downs even the patient does not notice. For example, based on the sound information (for example, voice information) acquired from the patient terminal device 20, the information processing device 40 compares, by using the analysis unit 41, the daily utterance with the utterance in normal times to determine emotional ups and downs of the patient (an example of the patient's symptom), and then generates emotion information (an example of the patient's symptom information) regarding the emotional ups and downs of the patient so as to be recorded in the storage unit 43. This emotion information is used as objective data. Note that the utterance in normal times is not limited to the one acquired for the first time. That is, it is possible to determine, during daily acquisition, an occurrence with a small change compared to the normal time as an occurrence in normal times, and constantly update the utterance data in normal times, thereby enabling more accurate determination of the occurrence in normal times and the utterance when an abnormality occurs.

In addition, it is also possible to use a nocturnal sleep state as an objective evaluation value. It is difficult for a patient asleep to recognize their symptoms. Therefore, in a case where the patient with a disease of asthma has a good sleep, it indicates that the symptom is eased, representing a good condition of the symptom. For example, using the detection unit 21 such as a camera and a vital signs sensor, the patient terminal device 20 measures patient's motion information and vital signs information such as a patient's body motion and pulse. Based on the patient's motion information and biometric information acquired from the patient terminal device 20, the information processing device 40 grasps, by using the analysis unit 41, the state where the patient is having a good sleep, generates sleep information regarding the patient's sleep, and records the generated information in the storage unit 43. This sleep information is used as objective data.

### Second Example of Objective Data

The patient terminal device 20 uses the detection unit 21 such as an environment sensor to detect and acquire information such as barometric pressure, the amount of dust and pollen in the air, temperature, and humidity, which are likely to affect a disease, as environmental information that is an example of objective data. Furthermore, the environment (surrounding environment of the patient) also changes depending on the location of the patient. Therefore, by acquiring position information (such as position information based on a global positioning system (GPS), for example) regarding the location of the patient and appropriately acquiring the environmental information at the target position, it is also possible to clarify the correlation between the environment and the disease. For example, depending on the location of the patient, there are various environments such as an environment with good air or an environment with bad air.

### 2-4. Subjective Data Related to Subjective Evaluation Values

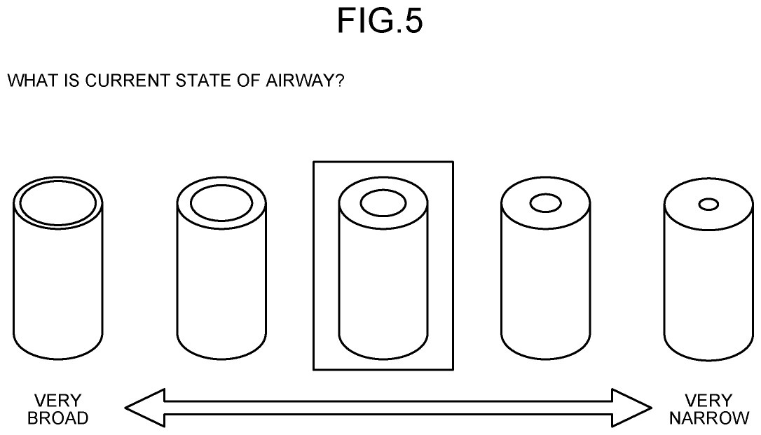

An example of subjective data related to the subjective evaluation value according to the present embodiment will be described with reference to FIGS. 5 to 8. FIGS. 5 to 8 are explanatory diagrams illustrating an example of subjective data acquisition according to the present embodiment.

The patient terminal device 20 acquires, by the input unit 22, a subjective symptom that is prominent for the disease and suitable for the user's sense. For example, in a case where the disease is asthma, the patient terminal device 20 guides the patient to input an image of the broadness of the airway. As an example, the patient terminal device 20 visualizes and presents a provisional state based on a numerical value obtained by acquiring a simple peak flow number, thereby allowing the patient to compare the provisional state with their sense and allowing the patient to correct the provisional state in accordance with their sense.

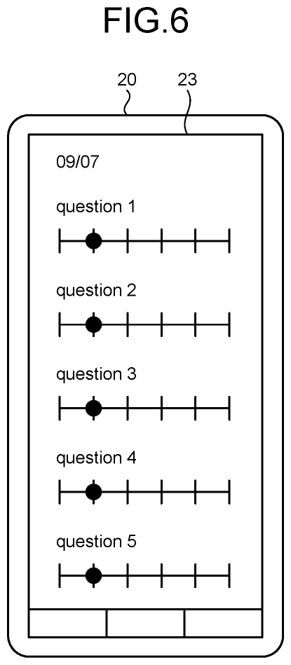

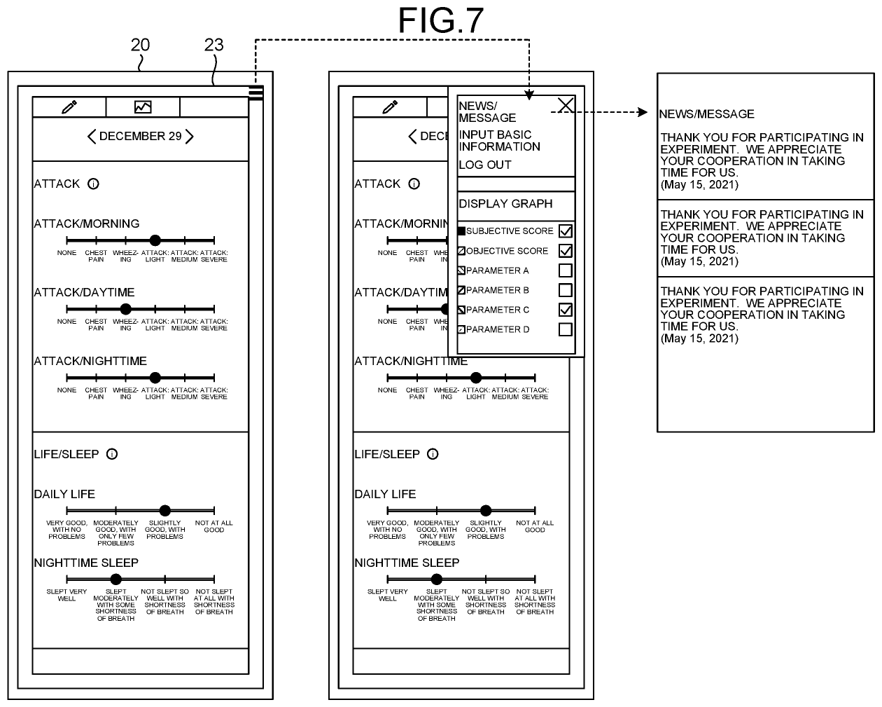

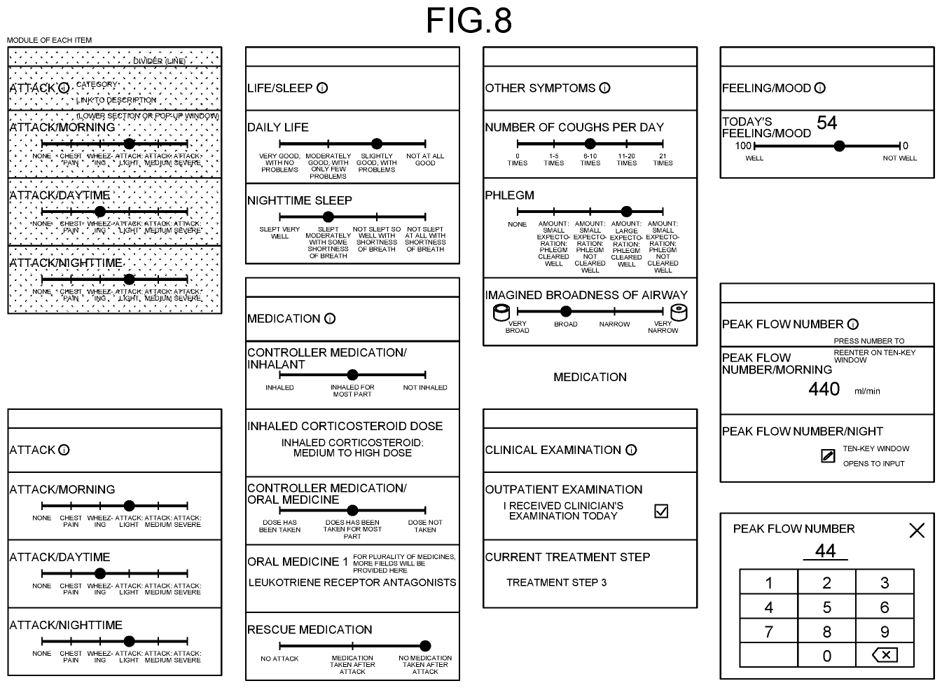

For example, it is allowable to use, as a graphical user interface (GUI) that is an example of an input screen, a GUI that prompts the patient to perform selection from among several examples of the size of the airway as illustrated in FIG. 5, or a GUI that prompts the patient to answer a level or the like felt by the patient for individual questions (medical inquiries) as illustrated in FIGS. 6 and 7. Individual questions, that is, items to be input by the patient include, as illustrated in FIG. 8, various input items (seizures, daily life/sleep, medication, other symptoms, medical care, emotion, and peak flow number, for example). These input items are sequentially or appropriately displayed according to an input operation such as scrolling or switching of a screen, for example.

In the example of FIG. 7, pressing the menu button located at the upper right of the input screen in FIG. 7 by the user will open a menu screen as another window, and further pressing “News/message” in the menu screen by the user will open the items as news/message. Such a display flow is merely an example, and other various display flows can be adopted.

It is also allowable to use, as another type of GUI, a GUI that displays a circle indicating the size of the airway and easily changes the size of the circle by pinching in/out or dragging a certain position. The default size of the airways, circles, and the like, can be determined based on a provisionally obtained peak flow number and it is allowable to use some method to clearly indicate a difference from the subjective evaluation of the patient.

The various GUIs function as images that enable the patient to select an answer to the medical inquiry, and also function as images for prompting the patient to input the broadness of the airway imagined. These GUIs are generated by the generation unit 44 of the information processing device 40 and transmitted to the patient terminal device 20 via the communication network 50. The patient terminal device 20 displays the transmitted GUI on the display unit 23. Alternatively, the patient terminal device 20 may generate and display the GUI.

Although the imagined size of the airway obtained as described above is different from the actual size of the airway, the imagined size is close to the symptom felt by the patient (subjective evaluation value). In addition, by presenting the relevance between the acquired size imagined and the peak flow number separately acquired, it is also possible to allow the patient to confirm the sense. In this case, the patient can be sensitive to their symptoms, making it possible to prompt the patient to take action to suppress the symptoms.

By the way, in the case of the currently used medical inquiry (PRO), the patient often looks back on one month at the time of medical examination occurring at a frequency such as once a month, and fills in the form while recalling the situation of the period. In this case, the patient's memory is indistinct, and thus the patient cannot be perfect in accurately inputting the symptoms.

To handle this, it is allowable to provide a GUI (an example of an image that prompts the patient to answer to the medical inquiry) that allows the patient to evaluate their symptoms while their memory is fresh, by performing comparison with the state of the previous day and check whether the patient feels better or worse than the previous day, or comparison with earlier times of the day, such as checking the state at night as compared with morning or daytime. Examples of this GUI include a GUI in which a message prompting an answer, various questions, and the like are added to the images illustrated in the drawings such as FIGS. 5, 6, and 7. Since subjective evaluation often fades away over time, it is useful to compare daily symptom inputs, as well as several times of (for example, three times in the morning, daytime, and night) symptom inputs per day, with previous inputs.

Moreover, by acquiring calendar information or behavior information as a clue for recalling the state of the day and displaying these pieces of information and the score image in association with each other, the patient can look back on the whole day by using the information as a trigger for recalling own symptoms. Note that an image indicating patient's behavior information or calendar information is displayed together with the score image.

Furthermore, for example, the patient may perform input operation on the input unit 22 of the patient terminal device 20 and input an answer to a question related to QoL in addition to the answer to the medical inquiry. The guidance at the time of inputting the answer may be provided, and in this case, the guidance may be provided by wording according to the patient's lifestyle or linguistic features. For example, the content, the ending, and the like may be adapted to the patient, and the speed may be adapted to the patient in the case of text-reading. These will make it possible to facilitate input of an answer to the question related to QoL.

### 2-5. Visualization of Objective Data and Subjective Data

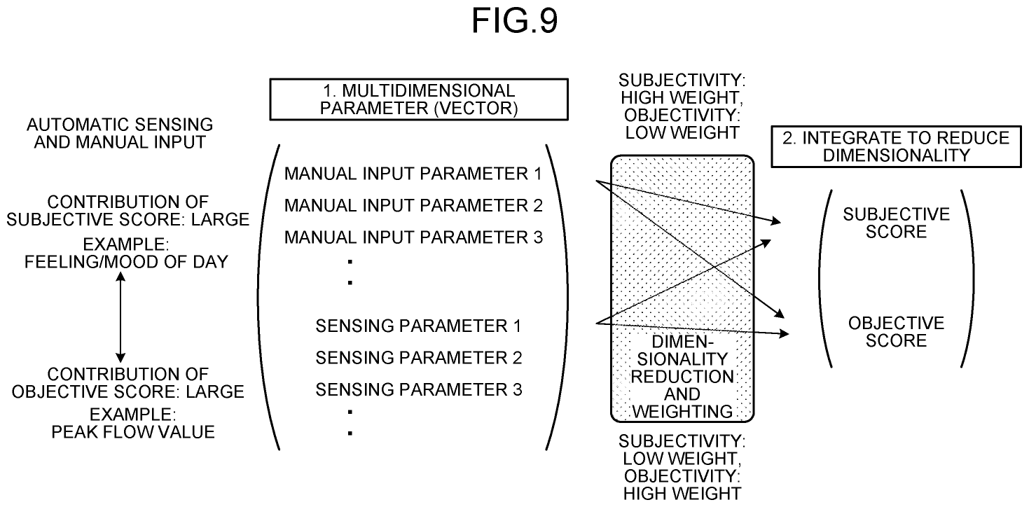

An example of visualization of objective data and subjective data according to the present embodiment will be described with reference to FIGS. 9 to 12. FIGS. 9 to 12 are explanatory diagrams illustrating an example of visualization of objective data and subjective data according to the present embodiment.

As illustrated in FIG. 9, the information processing device 40 uses the analysis unit 41 to integrate a plurality of pieces of objective data (for example, various data such as a pulse wave, a heart rate, a body temperature, and an expiratory volume (a numerical value of a peak flow)) acquired from the patient terminal device 20 into objective scores, and generates objective score data. When integrating the plurality of pieces of objective data such as sensing data into objective scores, the analysis unit 41 performs weighting according to the degree of importance of the objective data for each disease. For example, the analysis unit 41 multiplies each piece of objective data by a weighting factor, adds up the results, and integrates the results into objective scores. In a case where the chronic disease is asthma, in order to confirm the symptoms, the degree of importance is assumed to be in the order of: peak flow number>frequency of attack>quality of sleep . . . . In this case, the factor is adjusted by the degree of importance, and the objective score is obtained.

Furthermore, as illustrated in FIG. 9, in the information processing device 40 uses the processing unit 42 to integrate a plurality of pieces of subjective data (for example, various types of data such as a mood, a state of the airway, and the physical conditions of the day) acquired from the patient terminal device 20 into objective scores to generate subjective score data. When the plurality of pieces of subjective data such as the medical inquiry result data is integrated into the subjective score, the processing unit 42 performs weighting according to the degree of importance of the subjective data for each disease. For example, the processing unit 42 multiplies each piece of subjective data by a weighting factor, adds up the result of multiplication, and integrates the results into subjective scores. That is, by changing the weighting of the subjective score for each item depending on the disease, adjustment can be performed to reveal the subjective evaluation value of the target disease.

The weighting factor are set according to the patient's disease. However, since each clinician has mutually different determination criteria and may sometimes change the weighting factor accordingly, the weighting factor is set to be changeable by the clinician. In this case, the weighting factor is changed by operation on the input unit 31 of the clinician terminal device 30.

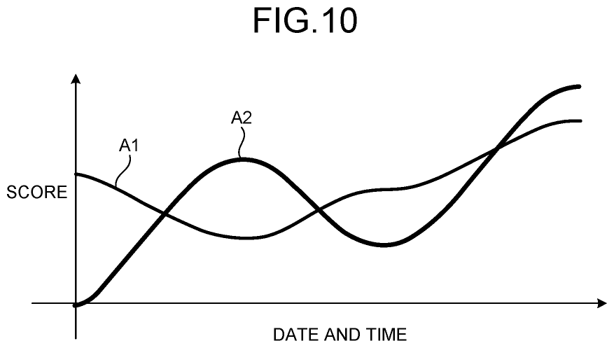

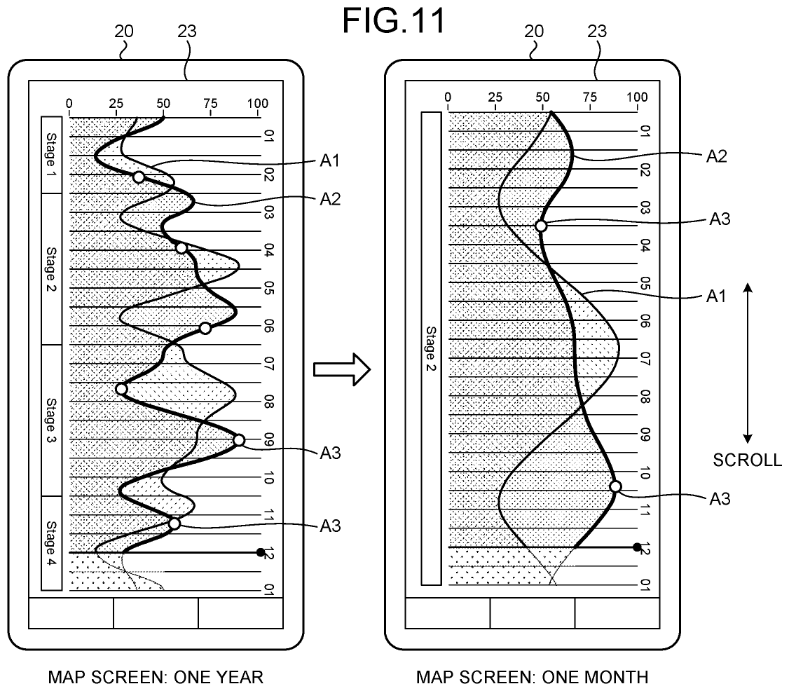

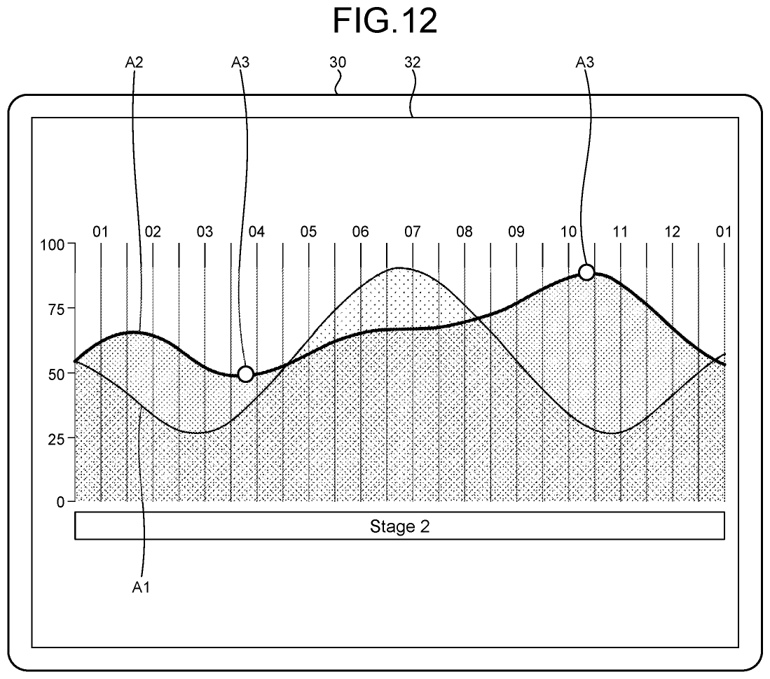

In addition, the information processing device 40 displays, in time series, the objective score and the subjective score generated by the generation unit 44 from individual pieces of subjective data individual pieces of objective data acquired from the patient terminal device 20. That is, as illustrated in FIG. 10, the generation unit 44 generates objective score data (objective score graph A1) indicating the objective scores in time series and subjective score data (subjective score graph A2) indicating the subjective scores in time series. Subsequently, based on the objective score data and the subjective score data, the generation unit 44 generates a score image including the objective score graph A1 and the subjective score graph A2 as illustrated in FIGS. 11 and 12.

The score image (for example, the map screen) is displayed on the display unit 23 of the patient terminal device 20 as illustrated in FIG. 11, or is displayed on the display unit 32 of the clinician terminal device 30 as illustrated in FIG. 12. In the example of FIG. 11, the map screen for one year and the map screen for one month are switchable. The period of the map screen can be appropriately changed, not limited to one year or one month.

Here, the scores displayed in time series are compared with preceding and subsequent values, and a portion having a large change or a portion having a significant difference between the objective score and the subjective score are indicated by using an image A3 (refer to FIGS. 11 and 12), which attracts attention of the clinician and the patient, so as to highlight the change point (an example of change information). In the examples of FIGS. 11 and 12, a circle is used as the image A3. As to what point is determined as the change point, a determination criterion is generated based on past patient data (including other patients).

Furthermore, by selecting an arbitrary point on the graph (objective score graph A1 or subjective score graph A2) of the score image, it is also possible to display detailed information of the point. For example, a detailed information screen, which displays the detailed information, displays an image indicating data such as source data of generation of the score of the point. This image may be displayed on a screen other than the screen currently displaying the score image or may be displayed superimposed on the score image.

### 2-6. Supporting Continuous Treatment

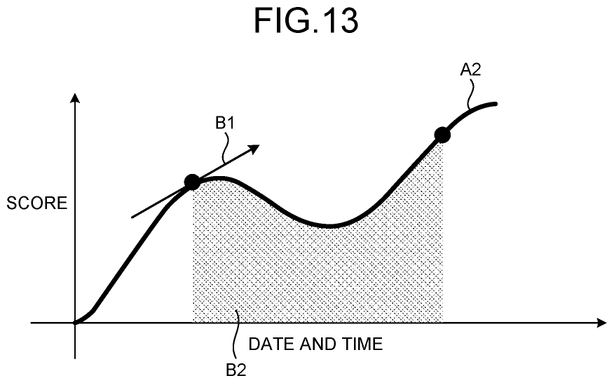

An example of continuous treatment according to the present embodiment will be described with reference to FIGS. 13 to 15. FIGS. 13 to 15 are explanatory diagrams illustrating an example of support of continuous treatment according to the present embodiment.

The clinician visually recognizes the score image (for example, a map) displayed by the display unit 32 of the clinician terminal device 30, and notifies the patient information such as an evaluation of the situation and the prospect of the symptom using the score image. These are comprehensively presented based on various types of information in the objective score data and the subjective score data, such as a relationship between score transition and symptom transition, a relationship between total score and score change up to the present point and symptom transition, and past symptom change in other patients.

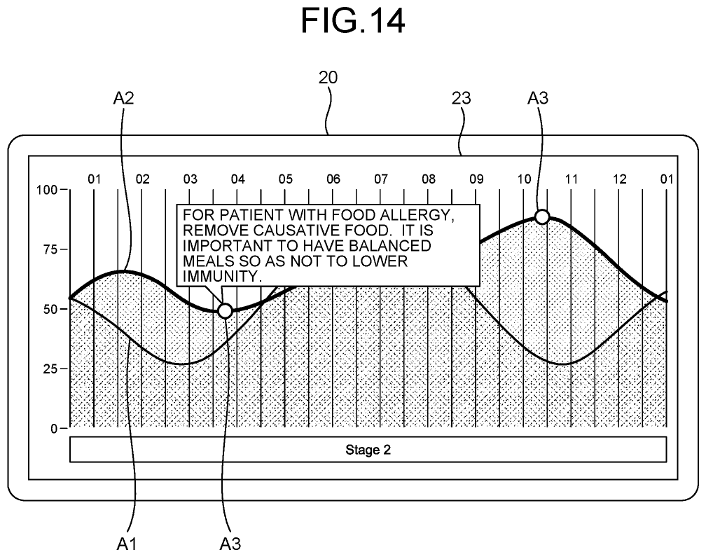

As illustrated in FIG. 13, for example, the steepness of an inclination B1 of the graph at a certain time point as well as positive/negative (+/−) are used for determination as the transition of the score. The total score is obtained by using a graph area, for example, the information processing device 40 can calculate an average score value by dividing the graph area by the number of days up to the present point, or extract a tendency in a certain period (a range B2 between certain time points) by cutting out the period. By comparing these score values with environment data, event information, or the like, it is possible to easily grasp the causal relationship between the presence or absence of environment or event and the change in score, enabling information useful for symptom control to be provided to the patient. For example, the information processing device 40 determines from past data that the symptoms tend to aggravate in a period having a large climate change, predicts a period in which the next climate change is likely to be large from weather information (forecast information) or the like, and displays, as a notification to the patient, an image indicating a message to promote health management when the period is approaching as illustrated in FIG. 14.

Furthermore, when the graph area approaches a certain threshold, the information processing device 40 can also predict the timing at which the threshold is exceeded based on environmental information, the latest symptom score, the schedule of the event, and the like, display a warning image to warn the patient. Furthermore, when the inclination of the graph is changed abruptly (when the inclination of the graph is changed greatly), a warning display can also be performed to present information useful for symptom control, or the point of great change can be appropriately focused on at the time of a consultation with the clinician to facilitate sharing of the situation.

Furthermore, by presenting an image indicating information for continuing treatment to the patient, the information processing device 40 may assist chronic disease treatment in which continuation of treatment is very important. For example, by presenting a state of symptom improvement using comparison of own data with input data of another patient so as to have a clear future prospect, it is possible to alleviate anxiety about a disease and to increase a treatment continuation rate and an application continuation rate. As the information for continuing the treatment, it is allowable to use, for example, information (such as annotation and message, for example) that motivates the patient to undergo the treatment.

### Specific Examples of Intervention

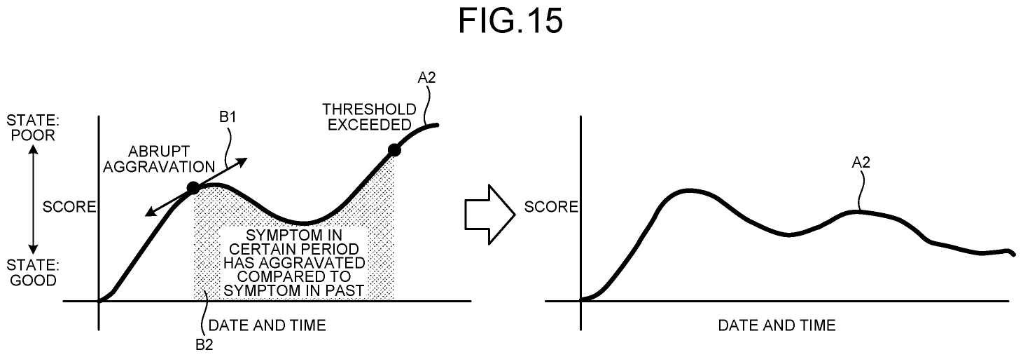

In the score image (for example, the map), as illustrated in FIG. 15, the graph (for example, the subjective score graph A2) is directed upward when the symptom is in a bad state, and the graph is directed downward when the symptom is in a good state. In a case where the inclination B1 of the graph exceeds a certain threshold or in a case where the score exceeds a certain threshold, an annotated image (an example of warning) may be presented to prompt the patient, the clinician, the medical worker, or the like to control the symptom. As in the example of FIG. 15, it is desirable to perform symptom control so that the graph is gradually directed downward and is stabilized at a constant value as much as possible.

As an example of the annotation, in a case where the graph is inclined upward, a specific suggestion is presented to the patient using an image indicating a message: “The symptom seems to be getting worse. Considering the future change in climate, be sure to refrain from exercise, take medicine, and get enough sleep”. In addition, a specific suggestion is presented to the clinician using an image indicating a message: “Although the patient's subjective score is in the right direction, the objective score exhibits a declining trend. It is worth suggesting re-examination of medicine at the time of consultation with the patient”. Having recognized the annotations, the clinician can use them as criteria for the determination of prescribing weaker medication when the patient's condition is improving and to prescribing a stronger medication when the patient's condition is declining.

In addition, it is also allowable to use other method other than just presenting an image indicating a message. Specifically, when the score exceeds a certain threshold or when the inclination of the graph exceeds a certain threshold, that is, when there is an abrupt change in the symptom, it is allowable to use an annotation enabling the news/message to reach the patient or the clinician even in a state where the patient is not viewing the score image, for example, a notification sound or light may be emitted. These annotations contribute to intervention to achieve a movement of the graph going downward as much as possible on the map and a reduced vertical movement of the wave.

### 2-7. Screen Example

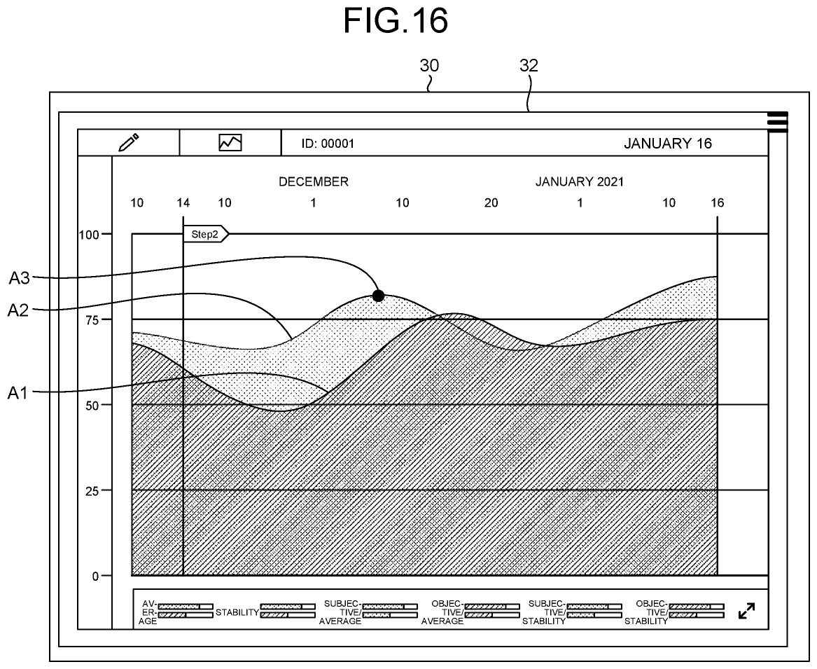

A screen example according to the present embodiment will be described with reference to FIGS. 16 to 20. FIGS. 16 to 20 are explanatory diagrams each illustrating an example of a screen according to the present embodiment. Various images (screens) according to each screen example may be displayed by the display unit 23 of the patient terminal device 20 or may be displayed by the display unit 32 of the clinician terminal device 30. In the examples of FIGS. 16 to 20, it is assumed that an image is displayed by the display unit 32 of the clinician terminal device 30.

As illustrated in FIG. 16, the display unit 32 can display a map image (map screen). The map image displays, for example, a patient identification/identifier (ID), which is an example of patient identification information, a menu button, a treatment stage, a graph (for example, an objective score graph A1, a subjective score graph A2, or the like), and a change point A3. The menu button is displayed at the upper right of the map image, and the treatment stage is displayed as “Step 2”. This makes it possible for the user to grasp various types of information.

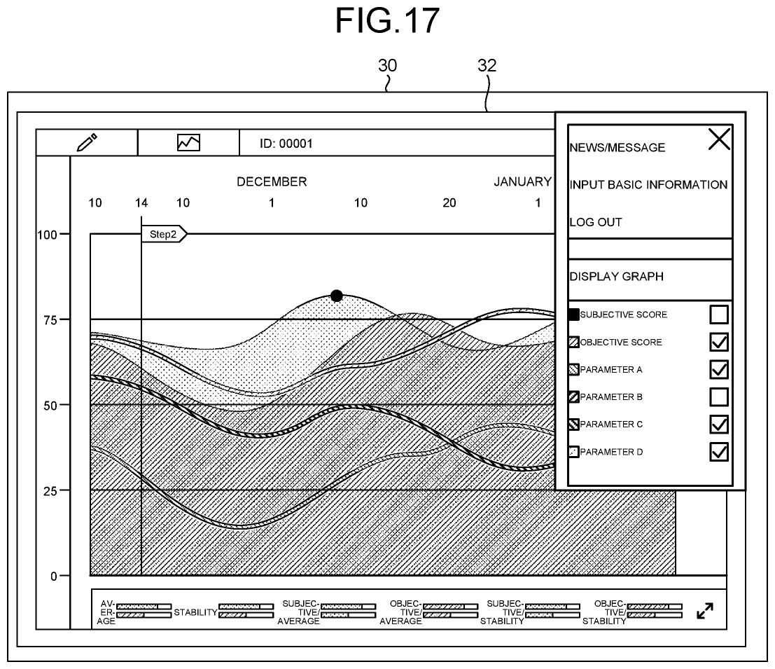

As illustrated in FIG. 17, the display unit 32 can display a map image and a menu image (menu screen). For example, when a menu button (refer to FIG. 16) is pressed by the user, the menu image is displayed to be superimposed on the map image. This menu image displays a plurality of checkboxes enabling selection of display of various graphs. When the checkbox is checked by the user, a graph of a parameter requiring detailed confirmation is displayed to be superimposed on the map image. This makes it possible for the user to confirm the graph of the parameter that requires detailed confirmation.

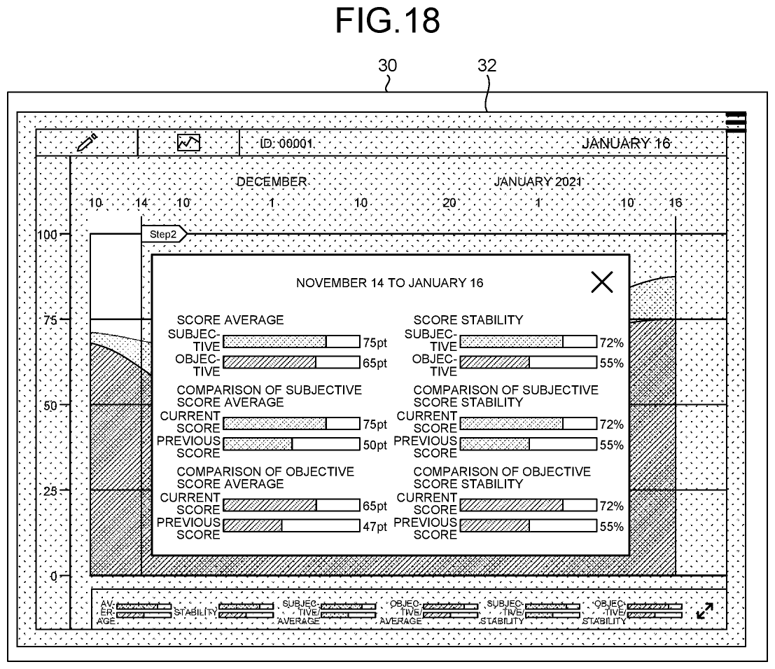

As illustrated in FIG. 18, the display unit 32 can display a horizontal bar chart image indicating the score average and the score stability in a certain period. For example, when a desired period on the graph of the map image is specified by the user, the horizontal bar chart image corresponding to the specified period is displayed to be superimposed on the map image. The horizontal bar chart image displays, for example, the average of the subjective and objective scores, the average of the current and previous subjective scores, the average of the current and previous objective scores, the stability of the subjective and objective scores, the stability of the current and previous subjective scores, and the stability of the current and previous objective scores in the specified period. Note that the average value may be obtained from the total score/number of days during the period, and the stability may be calculated as a value ranging 0-100 using a standard deviation or the like.

Such an example of FIG. 18 displays an image such as a horizontal bar chart image, that is, an image regarding a change in symptoms in a specific period on the graph. This makes it possible for the user to grasp the change in the symptom corresponding to the specific period. Furthermore, by displaying the average and the stability of each score as an example of the change in the symptom in the specific period, the user can confirm whether the patient can control the symptom in the period between medical examinations.

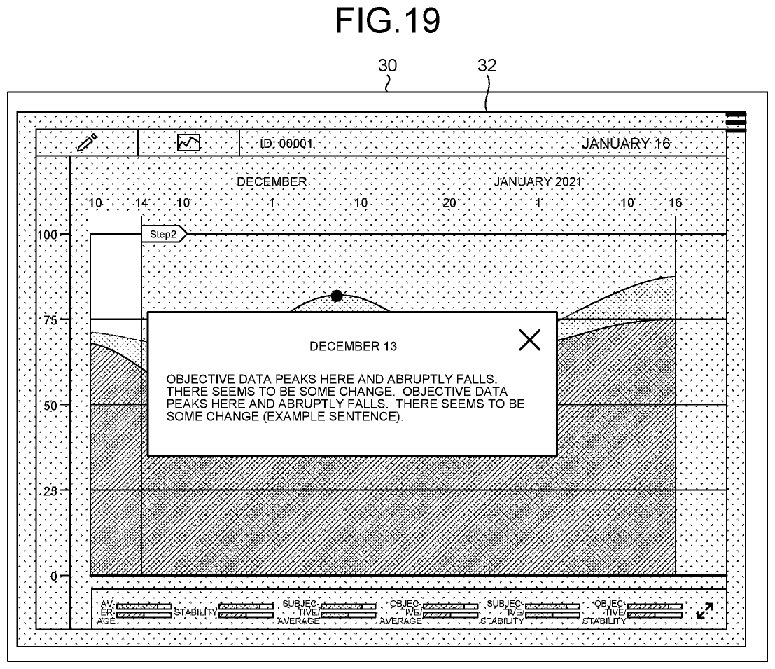

As illustrated in FIG. 19, the display unit 32 can display an explanatory image (explanation screen) illustrating explanation of a certain change point. For example, when a change point on the graph of the map image is selected by the user, an explanatory image indicating an explanatory sentence corresponding to the selected change point is displayed. For example, the explanatory sentence is an explanatory sentence as a basis for the selection of the extracted change point. This makes it possible for the user to confirm information regarding the certain change point.

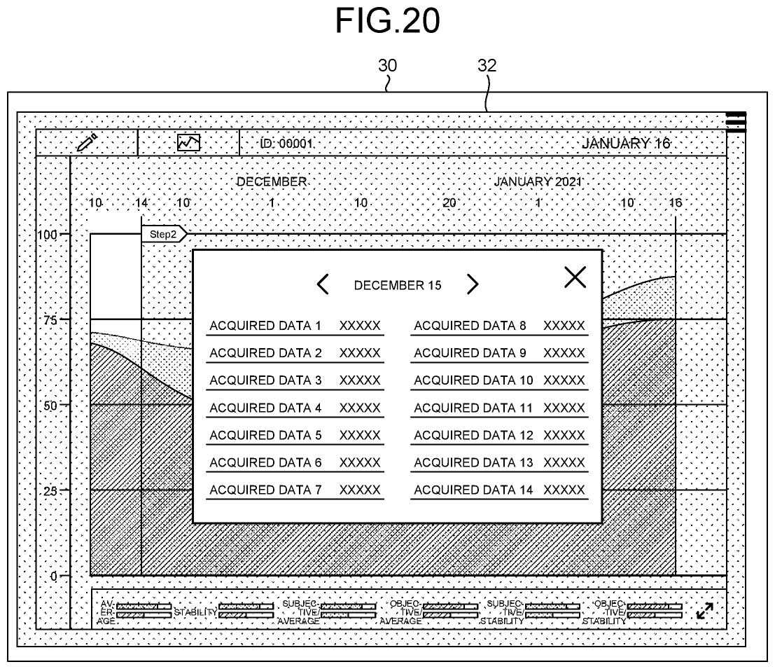

As illustrated in FIG. 20, the display unit 32 can display a detailed image (detailed screen) indicating detailed information of a certain date. For example, when a date on the graph of the map image is selected by the user, a detailed image indicating detailed information corresponding to the selected date is displayed. The example of FIG. 20 displays various types of acquired data (for example, acquired data 1 to 14) corresponding to the selected date and detailed images indicating detailed information of the acquired data. This makes it possible for the user to confirm various types of information corresponding to a certain date.

### 2-8. Medication Management

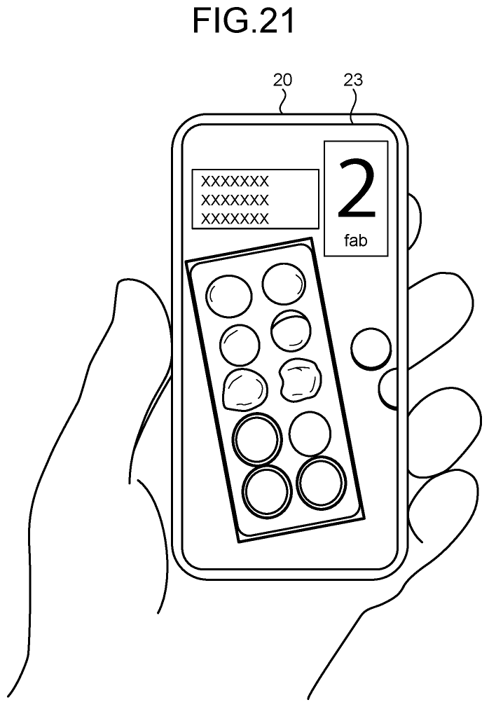

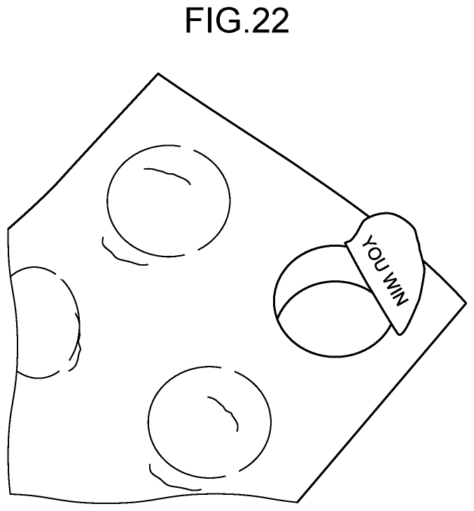

An example of medication management according to the present embodiment will be described with reference to FIGS. 21 and 22. FIGS. 21 and 22 are explanatory diagrams illustrating an example of medication management according to the present embodiment.

The information processing device 40 automatically acquires the medication state by the analysis unit 41 based on the objective data from the patient terminal device 20, and performs medication management by recording the obtained medication state in the storage unit 43. For example, in order to automatically acquire the medication state, the patient terminal device 20 uses the detection unit 21 such as a microphone and an acceleration sensor to detect data (including sound information or motion information when a patient takes a medicine, for example) such as an opening sound of a medicine package, a movement of a hand when taking a medicine, a sound of the throat when taking a medicine, and an inclination of the neck. The information processing device 40 uses the analysis unit 41 to determine whether the patient has taken the medicine (intake/non-intake of medication) based on the sound information or the motion information from the patient terminal device 20, and generates the information regarding the intake/non-intake of medication so as to be recorded in the storage unit 43. This information regarding the intake/non-intake of medication is used as objective data. Note that a medicine package having a characteristic easily detected as an opening sound of the medicine may be used.

Furthermore, as illustrated in FIG. 21, the patient terminal device 20 can also manually record the medication by capturing the image of the package of the medicine after taking the medicine by the detection unit 21 such as a camera. For example, based on data (for example, image information when a patient takes the medicine) such as an image of a package of a medicine captured by the patient terminal device 20, the information processing device 40 uses the analysis unit 41 to detect a change in the package of the medicine (whether the patient has taken the medicine), recognizes that the medicine has decreased (the patient has taken the medicine) based on the difference, and generates the information regarding the intake/non-intake of medication so as to be recorded in the storage unit 43. This information regarding the intake/non-intake of medication is used as objective data.

In a case where periodic medication is scheduled in the storage unit 43 or the like of the information processing device 40 and data is not recorded even at that time, it is determined that the patient has not taken the medication. In this case, it is possible to issue a warning (such as a message, for example) indicating non-intake of medication and prompt the patient to take the medication. This makes it possible to prevent missing of a dose and support continuation of treatment.

In addition, using a medicine package with modification to prompt the user's medication will also lead to the continuation of the treatment. For example, as illustrated in FIG. 22, a short message such as “You win” or “Good for you! Remembered to take the medicine!” may be printed on a package portion visible after taking out the medicine to motivate the patient to take medication. Examples of applicable messages include a support message, tips (for example, advice or the like) from a clinician or a pharmacist and a message to encourage each other collected from other patients taking the same medication.

Furthermore, the message may be provided to the patient terminal device 20 not only by the physical package of the medicine but also by an application or the like. For example, a message (an example of information for continuing treatment) may be displayed on the display unit 23 of the patient terminal device 20, or may be output by voice using a microphone or the like. At this time, the message may be automatically generated from a past message. Furthermore, in a case where the patient's behavior has changed in response to a certain message, the association between the message and the patient's behavior may be recorded, and a similar message may be presented to a patient in the same situation.

### Specific Examples of Other Diseases

Although the present embodiment is described mainly focusing on asthma as an example of target disease, application is not limited thereto. Application is also possible for chronic diseases such as allergic diseases, diabetes, and heart disease in a similar manner, that is, it is possible to prompt a patient, a clinician, and a medical worker to control symptoms by visualizing the subjective score and the objective score and presenting changes in both scores along time series.

In addition, heart disease requires continuous cardiac rehabilitation after onset of symptoms. The objective data applicable at this time include data such as physical activity level and the amount of exercise acquirable by a sensor such as a pedometer or a GPS, and data such as a heart rate, a pulse, and a blood oxygen level acquirable by vital signs sensing. Examples of the subjective data to be acquired include daily breathlessness, daily mood or feeling (feeling depressed or not, or having refreshed feeling or not, etc.) and the degree of load of exercise. The objective score and the subjective score may be calculated from these pieces of information and visualized by a graph. This makes it possible to share the symptoms at home between the medical worker and the patient during the cardiac rehabilitation by hospital visits. By appropriately grasping the symptoms and changes in the symptoms, it is possible to conduct consultation about the degree of application of the exercise load so as to relieve the symptoms and achieve an improvement in a right direction. In addition, by presenting an association between the degree of exercise load in the past and a change in the current score value or presenting a future exercise load plan and a score value change prediction, it is possible to make a prospect of symptom control, leading to a change in patient's consciousness and behavior.

In addition, dietary therapy is also part of important rehabilitation, and dietary therapy is also applicable to diseases such as diabetes. By acquiring a dietary record by an image or grasping the work in real time from a video or the like during cooking, it is also possible to give guidance about seasoning and ingredients on the spot. It is also possible to analyze the influence of the meal on the score value from the graph of the used menu of the meal and the visualized objective score and subjective score. This makes it possible to predict a score change in a case where a similar meal is taken continuously in the future, enabling determination of effectiveness of the method as an evidence for encouraging meal improvement.

### 2-9. Effects

As described above, according to the present embodiment, by providing the analysis unit 41 (an example of the first data generation unit) that generates objective score data indicating objective scores in time series based on a plurality of pieces of objective data regarding a patient, the processing unit 42 (an example of the second data generation unit) that generates subjective score data indicating subjective scores in time series based on a plurality of pieces of subjective data obtained from a patient, and the generation unit 44 (an example of the image generation unit) that generates a score image indicating the generated objective score data and the subjective score data, it is possible to generate the score image and display the generated score image on devices such as the patient terminal device 20 and the clinician terminal device 30. This visualizes both the objective score data and the subjective score data, making it possible for the clinician and the patient to achieve good mutual communication while visually recognizing each other's data. That is, it is possible to appropriately transmit information useful for symptom control to a patient, a clinician, a medical worker, and the like, leading to achievement of supporting continuation of treatment.

In addition, the analysis unit 41 obtains an objective score by multiplying each piece of the objective data by a weighting factor depending on a degree of importance of the each piece of objective data and summing up results of the multiplication, while the processing unit 42 obtains a subjective score by multiplying each piece of the subjective data by a weighting factor depending on a degree of importance of the each piece of data and summing up results of the multiplication. This makes it possible to appropriately obtain the objective score and the subjective score, leading to achievement of visualization of both the objective score data and the subjective score data by the display of the score images. As a result, it is possible to appropriately transmit information useful for symptom control to a patient, a clinician, a medical worker, and the like, leading to achievement of supporting continuation of treatment.

In addition, the above-described two weighting factors are set depending on the disease of the patient. This makes it possible to obtain an objective score and a subjective score in accordance with the disease of the patient.

Furthermore, the above-described two weighting factors are variable. This makes it possible for the clinician to adjust the weighting factor based on their own determination criterion, leading to acquisition of objective scores and the subjective scores according to the determination criterion of the clinician, for example.

In addition, the analysis unit 41 determines a symptom of the patient based on sound information occurring from the patient and generates symptom information of the patient as the objective data, or determines a sleep state of the patient and generates sleep information of the patient as the objective data. This makes it possible to obtain an objective score corresponding to the patient's symptom (for example, asthma symptom, mood swings, or the like) or sleep situation (for example, whether the patient has good sleep).

In addition, the analysis unit 41 determines intake/non-intake of medication by the patient based on sound information, motion information, or image information at a time of intake of the medication by the patient, and generates information regarding intake/non-intake of medication by the patient as the objective data. This makes it possible to obtain an objective score corresponding to the medication status (intake/non-intake of medication) of the patient. For example, the clinician can grasp the medication status of the patient, leading to prevention of the patient's missing of a dose, enabling supporting continuous treatment.

Furthermore, the generation unit 44 generates an image that enables the patient to select an answer to the medical inquiry. This makes it possible for the patient terminal device 20 to display an image that enables the patient to select an answer to the medical inquiry, helping the patient to easily answer to the medical inquiry.

The generation unit 44 may also generate an image for allowing the patient to input an imagined broadness of the airway. This makes it possible for the patient terminal device 20 to display an image for allowing the patient to input an imagined broadness of the airway, which helps the patient to easily answer the inquiry of the broadness of the airway imagined by themselves.

In addition, the generation unit 44 generates an image that prompts the patient to answer the medical inquiry. This makes it possible for the patient terminal device 20 to display an image that prompts the patient to answer to the medical inquiry, enabling the patient to reliably answer to the medical inquiry.

In addition, the generation unit 44 generates an image indicating patient's behavior information or calendar information in addition to an image prompting the patient to answer to the medical inquiry. With this operation, the image indicating the behavior information or the calendar information of the patient can be displayed by the patient terminal device 20 or the clinician terminal device 30, making it possible for the clinician or the patient to grasp the behavior information or the calendar information of the patient. For example, it is possible to allow the patient to look back on their behavior and give a trigger to recall their symptoms, enabling the patient to easily answer the medical inquiry.

In addition, one of the individual pieces of objective data is biometric information of the patient, and another piece of individual pieces of objective data is environmental information around the patient. With this configuration, not only the biometric information of the patient, but also the environmental information around the patient is to be used to obtain the objective score, making it possible to obtain the objective score corresponding to the environment around the patient. For example, when the patient's disease is asthma, the surrounding environment of the patient is important.

Further, one of the individual pieces of subjective data is information concerning answer to the medical inquiry to the patient. With this configuration, the information concerning answer is used to obtain the subjective score, making it possible to obtain the subjective score corresponding to the answer to the medical inquiry to the patient.

Moreover, the score image includes an image indicating both or one of a change point in the objective score data and a change point in the subjective score data. This makes it possible to display both or one of the change point of the objective score data and the change point of the subjective score data together with the score image, enabling notifying the clinician and the patient of the change point of each data.

In addition, the score image includes an image indicating detailed information related to the objective score included in the objective score data or to the subjective score included in the subjective score data. This makes it possible to display the detailed information related to the objective score or the subjective score together with the score image, enabling notifying the clinician or the patient of the detailed information related to the objective score or the subjective score.

In addition, the score image includes an image indicating a message directed to the patient. This makes it possible to display the message directed to the patient together with the score image, enabling notifying the patient of the message. This leads to successful support of continuation of treatment.

In addition, the score image includes an image indicating information for encouraging the patient to continue the treatment. This makes it possible to display the information for encouraging the patient to continue the treatment together with the score image, enabling notifying the patient of the information for encouraging the patient to continue the treatment. This leads to successful support of continuation of treatment.

In addition, the score image includes an image indicating a message directed to the clinician. This makes it possible to display the message directed to the clinician together with the score image, enabling notifying the clinician of the message. For example, the clinician can perform appropriate treatment according to the message, leading to achievement of successful symptom control.

The generation unit 44 generates a graph as a score image, and the score image includes an image indicating a warning corresponding to an inclination of the graph at a certain time point. This makes it possible to display the warning together with the score image, enabling notifying the clinician and the patient of the warning.

In addition, the generation unit 44 generates a graph as a score image, and the score image includes an image indicating a warning corresponding to an area of the graph within a range between certain time points. This makes it possible to display the warning together with the score image, enabling notifying the clinician and the patient of the warning.

### 3. Other Embodiments

The processing according to the above-described embodiments (or modifications) may be performed in various different forms (modifications) other than the above-described embodiments. For example, among each process described in the above embodiments, all or a part of the processes described as being performed automatically may be manually performed, or the processes described as being performed manually can be performed automatically by a known method. In addition, the processing procedures, specific names, and information including various data and parameters illustrated in the above Literatures or drawings can be arbitrarily altered unless otherwise specified. For example, a variety of information illustrated in each of the drawings are not limited to the information illustrated.

In addition, each of components of each device is provided as a functional and conceptional illustration and thus does not necessarily need to be physically configured as illustrated. That is, the specific form of distribution/integration of each device is not limited to those illustrated in the drawings, and all or a part thereof may be functionally or physically distributed or integrated into arbitrary units according to various loads and use conditions.

Furthermore, the above-described embodiments (or modifications) can be appropriately combined within a range implementable without contradiction of processes. The effects described in the present specification are merely examples, and thus, there may be other effects, not limited to the exemplified effects.

### 4. Hardware Configuration Example

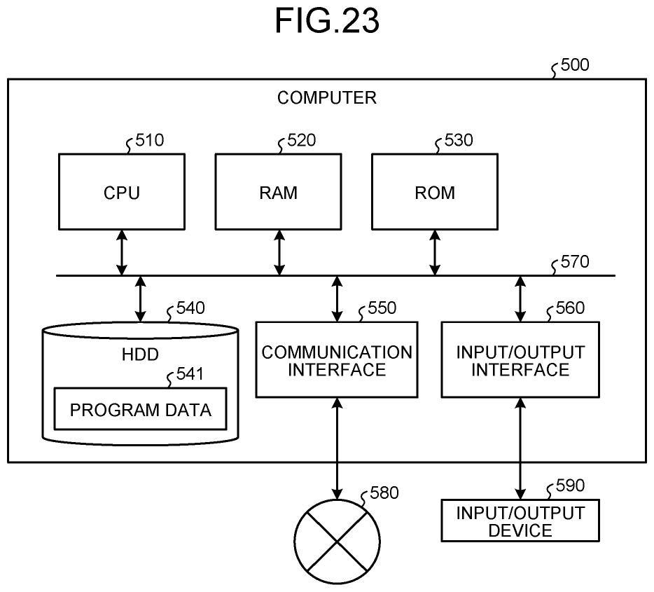

A specific hardware configuration example of information devices such as the patient terminal device 20, the clinician terminal device 30, and the information processing device 40 according to the above-described embodiment (or modification) will be described. The information devices such as the patient terminal device 20, the clinician terminal device 30, and the information processing device 40 according to the embodiment (or the modification) may be implemented by a computer 500 having a configuration as illustrated in FIG. 23, for example. FIG. 23 is a diagram illustrating a configuration example of hardware that implements functions of information devices such as the patient terminal device 20, the clinician terminal device 30, and the information processing device 40 according to the embodiment (or modification).

As illustrated in FIG. 23, the computer 500 includes a CPU 510, RAM 520, read only memory (ROM) 530, a hard disk drive (HDD) 540, a communication interface 550, and an input/output interface 560. Individual components of the computer 500 are interconnected by a bus 570.

The CPU 510 operates based on a program stored in the ROM 530 or the HDD 540 so as to control each of components. For example, the CPU 510 develops a program stored in the ROM 530 or the HDD 540 to the RAM 520, and executes processing corresponding to various programs.

The ROM 530 stores a boot program such as a basic input output system (BIOS) executed by the CPU 510 when the computer 500 starts up, a program dependent on hardware of the computer 500, or the like.

The HDD 540 is a non-transitory computer-readable recording medium that records a program executed by the CPU 510, data used by the program, or the like. Specifically, the HDD 540 is a recording medium that records an information processing program according to the present disclosure, which is an example of program data 541.

The communication interface 550 is an interface for connecting the computer 500 to an external network 580 (for example, the Internet). For example, the CPU 510 receives data from other devices or transmits data generated by the CPU 510 to other devices via the communication interface 550.

The input/output interface 560 is an interface for connecting an input/output device 590 with the computer 500. For example, the CPU 510 receives data from an input device such as a keyboard or a mouse via the input/output interface 560. In addition, the CPU 510 transmits data to an output device such as a display, a speaker, or a printer via the input/output interface 560.

Furthermore, the input/output interface 560 may function as a media interface for reading a program or the like recorded on a predetermined recording medium. Examples of the media include optical recording media such as a digital versatile disc (DVD) or a phase change rewritable disk (PD), a magneto-optical recording medium such as a magneto-optical disk (MO), a tape medium, a magnetic recording medium, and semiconductor memory.

Here, for example, in a case where the computer 500 functions as the information processing device 40 according to the embodiment, the CPU 510 of the computer 500 executes an information processing program loaded on the RAM 520, thereby implementing all or some of the functions of the analysis unit 41, the processing unit 42, the storage unit 43, the generation unit 44, and the like. In addition, the HDD 540 stores an information processing program and data (for example, objective data, subjective data, objective score data, subjective score data, score image, and the like) according to the present disclosure. While the CPU 510 executes program data 541 read from the HDD 540, the CPU 510 may acquire these programs from another device via the external network 580, as another example.

### 5. Supplementary Notes

Note that the present technique can also have the following configurations.

(1)

An information processing device comprising:
a first data generation unit that generates objective score data, which indicates objective scores in time series, based on a plurality of pieces of objective data regarding a patient;
a second data generation unit that generates subjective score data, which indicates subjective scores in time series, based on a plurality of pieces of subjective data obtained from the patient; and
an image generation unit that generates a score image indicating the objective score data and the subjective score data.

(2)

The information processing device according to (1),
wherein the first data generation unit obtains each of the objective scores by multiplying each of the plurality of pieces of objective data by a weighting factor depending on a degrees of importance of the each of the plurality of pieces of objective data and summing up results of the multiplication, and
the second data generation unit obtains each of the subjective scores by multiplying each of the plurality of pieces of subjective data by a weighting factor depending on a degrees of importance of the each of the plurality of pieces of subjective data and summing up results of the multiplication.

(3)

The information processing device according to (2),
wherein the two types of weighting factors are set depending on a disease of the patient.

(4)

The information processing device according to (2) or (3),
wherein the two types of weighting factors are variable.

(5)

The information processing device according to any one of (1) to (4),
wherein the first data generation unit determines a symptom of the patient based on sound information occurring from the patient and generates symptom information of the patient as the objective data, or determines a sleep state of the patient and generates sleep information of the patient as the objective data.

(6)

The information processing device according to any one of (1) to (5),
wherein the first data generation unit determines intake/non-intake of medication by the patient based on sound information, motion information, or image information at a time of intake of the medication by the patient, and generates information regarding intake/non-intake of medication by the patient as the objective data.

(7)

The information processing device according to any one of (1) to (6),
wherein the image generation unit generates an image that enables the patient to select an answer to a medical inquiry.

(8)

The information processing device according to any one of (1) to (7),
wherein the image generation unit generates an image that prompts the patient to answer a medical inquiry.

(9)

The information processing device according to (8),
wherein the image generation unit generates an image indicating behavior information or calendar information of the patient.

(10)

The information processing device according to any one of (1) to (9),
wherein one of the plurality of pieces of objective data is biometric information of the patient, and
another piece of the plurality of pieces of objective data is environmental information around the patient.

(11)

The information processing device according to any one of (1) to (10),
wherein one of the plurality of pieces of subjective data is information concerning answer to the medical inquiry to the patient.

(12)

The information processing device according to any one of (1) to (11),
wherein the score image includes an image indicating both or one of a change point of the objective score data and a change point of the subjective score data.

(13)

The information processing device according to any one of (1) to (12),
wherein the score image includes an image indicating detailed information related to the objective score included in the objective score data or to the subjective score included in the subjective score data.

(14)

The information processing device according to any one of (1) to (13),
wherein the score image includes an image indicating a message directed to the patient.

(15)

The information processing device according to any one of (1) to (14),
wherein the score image includes an image indicating information for encouraging the patient to continue treatment.

(16)

The information processing device according to any one of (1) to (15),
wherein the score image includes an image indicating a message directed to a clinician.

(17)

The information processing device according to any one of (1) to (16),
wherein the image generation unit generates a graph as the score image, and
the score image includes an image indicating a warning corresponding to an inclination of the graph at a certain time point.

(18)

The information processing device according to any one of (1) to (17),
wherein the image generation unit generates a graph as the score image, and
the score image includes an image indicating a warning corresponding to an area of the graph within a range between certain time points.

(19)

An information processing method performed by a computer, the method comprising:
generating objective score data, which indicates objective scores in time series, based on a plurality of pieces of objective data regarding a patient;
generating subjective score data, which indicates subjective scores in time series, based on a plurality of pieces of subjective data obtained from the patient; and
generating a score image indicating the objective score data and the subjective score data.

(20)

An information processing system comprising:
a patient terminal device that transmits a plurality of pieces of objective data regarding a patient and a plurality of pieces of subjective data obtained from the patient;
a first data generation unit that generates objective score data, which indicates objective scores in time series, based on the plurality of pieces of objective data transmitted by the patient terminal device;
a second data generation unit that generates subjective score data, which indicates subjective scores in time series, based on the plurality of pieces of subjective data transmitted by the patient terminal device;
an image generation unit that generates a score image indicating the objective score data and the subjective score data; and
a display unit that displays the score image generated by the image generation unit.

(21)

An information processing method using the information processing device according to any one of (1) to (18).

(22)

An information processing system including the information processing device according to any one of (1) to (18).

### REFERENCE SIGNS LIST

10 INFORMATION PROCESSING SYSTEM
20 PATIENT TERMINAL DEVICE
21 DETECTION UNIT
22 INPUT UNIT
23 DISPLAY UNIT
30 CLINICIAN TERMINAL DEVICE
31 INPUT UNIT
32 DISPLAY UNIT
40 INFORMATION PROCESSING DEVICE
41 ANALYSIS UNIT
42 PROCESSING UNIT
43 STORAGE UNIT
44 GENERATION UNIT
50 COMMUNICATION NETWORK

1. An information processing device comprising:
a first data generation unit that generates objective score data, which indicates objective scores in time series, based on a plurality of pieces of objective data regarding a patient;
a second data generation unit that generates subjective score data, which indicates subjective scores in time series, based on a plurality of pieces of subjective data obtained from the patient; and
an image generation unit that generates a score image indicating the objective score data and the subjective score data.

2. The information processing device according to claim 1,
wherein the first data generation unit obtains each of the objective scores by multiplying each of the plurality of pieces of objective data by a weighting factor depending on a degrees of importance of the each of the plurality of pieces of objective data and summing up results of the multiplication, and
the second data generation unit obtains each of the subjective scores by multiplying each of the plurality of pieces of subjective data by a weighting factor depending on a degrees of importance of the each of the plurality of pieces of subjective data and summing up results of the multiplication.

3. The information processing device according to claim 2,
wherein the two types of weighting factors are set depending on a disease of the patient.

4. The information processing device according to claim 2,
wherein the two types of weighting factors are variable.

5. The information processing device according to claim 1,
wherein the first data generation unit determines a symptom of the patient based on sound information occurring from the patient and generates symptom information of the patient as the objective data, or determines a sleep state of the patient and generates sleep information of the patient as the objective data.

6. The information processing device according to claim 1,
wherein the first data generation unit determines intake/non-intake of medication by the patient based on sound information, motion information, or image information at a time of intake of the medication by the patient, and generates information regarding intake/non-intake of medication by the patient as the objective data.

7. The information processing device according to claim 1,
wherein the image generation unit generates an image that enables the patient to select an answer to a medical inquiry.

8. The information processing device according to claim 1,
wherein the image generation unit generates an image that prompts the patient to answer a medical inquiry.

9. The information processing device according to claim 8,
wherein the image generation unit generates an image indicating behavior information or calendar information of the patient.

10. The information processing device according to claim 1,
wherein one of the plurality of pieces of objective data is biometric information of the patient, and
another piece of the plurality of pieces of objective data is environmental information around the patient.

11. The information processing device according to claim 1,
wherein one of the plurality of pieces of subjective data is information concerning answer to the medical inquiry to the patient.

12. The information processing device according to claim 1,
wherein the score image includes an image indicating both or one of a change point of the objective score data and a change point of the subjective score data.

13. The information processing device according to claim 1,
wherein the score image includes an image indicating detailed information related to the objective score included in the objective score data or to the subjective score included in the subjective score data.

14. The information processing device according to claim 1,
wherein the score image includes an image indicating a message directed to the patient.

15. The information processing device according to claim 1,
wherein the score image includes an image indicating information for encouraging the patient to continue treatment.

16. The information processing device according to claim 1,
wherein the score image includes an image indicating a message directed to a clinician.

17. The information processing device according to claim 1,
wherein the image generation unit generates a graph as the score image, and
the score image includes an image indicating a warning corresponding to an inclination of the graph at a certain time point.

18. The information processing device according to claim 1,
wherein the image generation unit generates a graph as the score image, and
the score image includes an image indicating a warning corresponding to an area of the graph within a range between certain time points.

19. An information processing method performed by a computer, the method comprising:
generating objective score data, which indicates objective scores in time series, based on a plurality of pieces of objective data regarding a patient;
generating subjective score data, which indicates subjective scores in time series, based on a plurality of pieces of subjective data obtained from the patient; and
generating a score image indicating the objective score data and the subjective score data.

20. An information processing system comprising:
a patient terminal device that transmits a plurality of pieces of objective data regarding a patient and a plurality of pieces of subjective data obtained from the patient;
a first data generation unit that generates objective score data, which indicates objective scores in time series, based on the plurality of pieces of objective data transmitted by the patient terminal device;
a second data generation unit that generates subjective score data, which indicates subjective scores in time series, based on the plurality of pieces of subjective data transmitted by the patient terminal device;
an image generation unit that generates a score image indicating the objective score data and the subjective score data; and
a display unit that displays the score image generated by the image generation unit.
